(19) United States
(12) Patent Application Publication
(10) Pub. No.: US 2010/0284986 A1
(43) Pub. Date: Nov. 11, 2010

(54) COMPOSITIONS AND METHODS FOR PREVENTION AND TREATMENT OF MIGRAINES

(76) Inventor: Kevin J. Kelleher, Herndon, VA (US)

Correspondence Address: PATTON BOGGS LLP, 8484 WESTPARK DRIVE, SUITE 900, MCLEAN, VA, 22102, US

(21) Appl. No.: 12/774,536

(22) Filed: May 5, 2010

Related U.S. Application Data
(60) Provisional application No. 61/175,893, filed on May 6, 2009.

(51) Int. Cl. A61K 36/28; A61P 25/06

(52) U.S. Cl. 424/94.1

(57) ABSTRACT

A therapeutic composition for preventing migraines is described. The therapeutic composition may include riboflavin, magnesium, feverfew and coenzyme Q10.

### CROSS-REFERENCE TO RELATED APPLICATION

This application claims the benefit of U.S. Provisional Patent Application No. 61/175,893, filed May 6, 2009, the contents of which are incorporated by reference herein in its entirety.

### FIELD OF THE INVENTION

The present invention relates generally to therapeutic compositions and methods, and more particularly, to therapeutic compositions and methods for prevention and treatment of migraines.

### BACKGROUND OF THE INVENTION

Migraines are a common, debilitative and chronic disease suffered by approximately 25-30 million individuals in the United States alone. The lifetime prevalence of migraines is around 12-16%, but varies by age and gender. For example, the percentages are approximately 18.2% for women and approximately 6.5% for men. Between ages 30-39, approximately 1 in 4 women and approximately 1 in 10 men suffer from migraines. The highest prevalence of migraines is in individuals between 30 and 49 years of age. Women in their 30s tend to have increased severity as well as increased frequency of migraines. Emerging evidence indicates that migraine may be a chronic progressive disorder characterized by escalating frequency of headache attacks.

Nine out of 10 migraine sufferers report they cannot “function normally” during days in which a migraine strikes, and nearly 3 in 10 require bed rest. More than 25% of migraine sufferers missed at least one day of work over the past three months due to a migraine. Approximately 50-75% of migraine sufferers report that migraines prevented them from doing household chores. Approximately 30-60% of migraine sufferers report that they did not participate in a family or social activity due to a migraine.

Migraines detrimentally impact the daily lives of migraine sufferers. Approximately 25% of migraine suffers experience four or more attacks a month. The median duration for a migraine attack is about 9-24 hours, but about 12% of migraines last longer than 24 hours.

Migraines are distinct from regular headaches. Migraines are believed to be of a neurogenic origin and involve a hypersensitivity of the nervous system. A migraine generator area of the brainstem is activated, initiating a wave of neuronal depression across the cortex and then triggering other brain centers and vascular structures. This process results in significant disability from the migraine as well as the frequently associated symptoms of nausea, light sensitivity, noise sensitivity, fatigue, neck pain, mood disturbances, and others. These symptoms can be experienced for longer than 72 hours and some can persist even after the migraine has resolved. As noted above, these symptoms result in significant disability for the migraine sufferer.

Nearly all prevention medications used by physicians are drugs developed for other reasons but have been shown to be helpful for migraines. Unfortunately, the potential side effects and drug interactions possible for these blood pressure drugs, anti-depression drugs, anti-psychotic drugs and anti-seizure drugs are significant. Non-prescription drug options are often unstudied or used at ineffective doses.

There is significant need for an accessible, well-tolerated and convenient compositions for migraine sufferers to use in treating migraines and/or reducing or eliminating the occurrence of migraines.

### SUMMARY OF THE INVENTION

Accordingly, the present invention provides a therapeutic composition for preventing migraines. The therapeutic composition may include riboflavin, magnesium, feverfew and coenzyme Q10.

It is to be understood that both the foregoing general description and the following detailed description are exemplary and explanatory and are not intended to limit the invention as claimed. Other objects and features of the invention will become apparent from the following detailed description. All references cited in the instant disclosure are incorporated herein by reference.

### DETAILED DESCRIPTION

Embodiments of the present invention may include migraine prevention compositions, although use for prevention of common headaches, tension headaches, trigeminal system-based headaches, including cluster headaches, may be considered. In certain embodiments, therapeutic compositions may include riboflavin, magnesium, feverfew and/or coenzyme Q10 (CoQ-10). Ranges of therapeutic compositions may include, per day: approximately 100-400 mg riboflavin, approximately 150-600 mg magnesium, approximately 25-100 mg feverfew, and approximately 75-1200 mg coenzyme Q10, preferably delivered as twice a day dosing. In preferred embodiments, the therapeutic compositions may contain, per dose, approximately 200 mg riboflavin, approximately 150 mg magnesium, approximately 25 mg feverfew, and approximately 75 mg CoQ-10. Compositions may additionally include carnitine, essential fatty acids, EPA or DHA, selenium, zinc, 5HTP, various analgesics or pain relievers, MSM, melatonin, lysine, GABA ands its variations, epilepsy drugs such as valproic acid, valerian root, ginger, butterbur, black cohosh, and combinations of these additives.

Riboflavin, also known as vitamin B2, is an easily absorbed micronutrient with a key role in maintaining health in humans and animals. It is the central component of the cofactors required by all flavoproteins. As such, riboflavin is required for a wide variety of cellular processes. Migraine pathogenesis may involve mitochondrial dysfunction that results in impaired oxygen metabolism. The brains of migraine headache sufferers are characterized between attacks by a reduction of mitochondrial phosphorylation potential. Riboflavin, which has the potential of increasing mitochondrial energy efficiency, has a prophylactic effect in treating migraine headaches.

Low magnesium levels have been demonstrated in the red blood cells and saliva of migraine sufferers. Reduced intracellular concentrations of magnesium have also been shown in the cortex of migraine subjects using ³¹P-magnetic resonance spectroscopy (P-MRS). Both during and in between attacks, cerebrospinal fluid levels of magnesium are significantly lower in migraine patients compared to normal patients. Thus, magnesium may be involved in the occurrence of migraines. In vitro and human studies have shown that low magnesium has several effects that may lead to the occurrence of migraine. Low magnesium levels induce cerebral arterial vasospasm and potentiate a contractile response of blood vessels to vasoactive substances such as serotonin. Magnesium also enhances the sensitivity of N-methyl D-aspartate (NMDA) receptors to glutamate, thus inducing epileptiform discharges and cortical spreading depression. Pro-inflammatory effects have also been observed with increases in platelet aggression leading to serotonin release. Hence, supplementation of the therapeutic composition of the present invention with magnesium may provide some relief for migraine sufferers. Administration of magnesium may reduce the frequency of migraine headaches.

Magnesium may be in the form of magnesium salts. Magnesium salts may include magnesium chloride, magnesium citrate, magnesium gluceptate, magnesium gluconate, magnesium hydroxide, magnesium lactate, magnesium oxide, magnesium sulfate, magnesium lactate, magnesium pidolate, magnesium carbonate, and magnesium tartarate.

Feverfew may be used in the present invention for reducing fever, for treating headaches, arthritis and digestive problems. It is hypothesized that by inhibiting the release of serotonin and prostaglandins, both of which are believed to aid the onset of migraines, feverfew limits the inflammation of blood vessels in the head. This may stop the blood vessel spasm, which is believed to contribute to headaches. The active ingredients in feverfew include parthenolide and tanetin.

CoQ-10 is an oil-soluble vitamin-like substance that is present in most eukaryotic cells, primarily in the mitochondria. It is a component of the electron transport chain and participates in aerobic cellular respiration, generating energy in the form of ATP. CoQ-10 may reduce the frequency of migraine attacks.

In certain embodiments, a user may take one or more doses per day. The composition may be recommended as twice a day dosing, although the composition may also be recommended for daily dosing. Therapeutic response, defined as reduction in severity or frequency of headaches, may allow for eradication in as little as approximately 1-2 weeks, although it may take 6 weeks or longer for response to be observed in some circumstances. Once effective, the dosing regimen may be continued for approximately 3-6 months, as per current medical guidelines for prevention.

The therapeutic composition is preferably administered orally, but other administration routes may be possible. Dosage forms may include one or more of a caplet, tablet, gel capsule, powder capsule, gel, liquid, or powder. Variations of the dose may be available when appropriate for other comorbid conditions. Non-active ingredients, such as, but not limited to, fillers, anti-caking agents, lubricants, preservatives, may be added to the dosage form.

The compositions and methods of the present invention may decrease or eliminate frequency of migraines, thereby ameliorating migraine-related disabilities. In certain embodiments, the therapeutic compositions may be reformulated with various other supplements or drugs already in use for prevention and treatment of migraines. The therapeutic compositions may be combined with various supplements, vitamins, minerals or other active ingredients. Additives may include, but are not limited to, folates or folate metabolites or derivatives, carnitine, essential fatty acids, EPA or DHA, N-acetyl cysteine, selenium, potassium, calcium, zinc, various vitamins such as niacin, niacinamide, the various forms of vitamin B-6 (such as pyridoxal-5-phosphate (P-5-P)), the various forms of vitamin B-12 (cyanocobalamin, methylcobalamin, hydroxycobalamin, etc.) and vitamin C and metabolites such as dehydroascorbic acid, ergotamines, 5 HTP, mast cell stabilizers, various analgesic or pain relievers such as aspirin, ibuprofen, and acetaminophen, channel blockers, beta blockers, i-NOs inhibitors (inducible nitric oxide inhibitors), NMDA antagonists (for example ketarnine, memantine, DL-2-aminophosphonovaleric acid (APV) and NAC), melatonin, taurine, homo-taurine, lysine, GABA and the nicotinate form of GABA (GABA(nictinoyl)), epilepsy drugs such as Valproate, compounds which increase GABA levels, glycine, progesterone, DLPA (D,L-phenylalanine) D-phenylal anine and L-phenylalanine, quercetin, histamine, isoflavones, curcumin and metabolites and derivatives thereof, aminoguanidine, L-carnosine, indoles, isothiocyanates and derivatives thereof, corydalis (dl-tetrahydropalmatine), and various natural products, such as valerian root, ginger, butterbur, nettle leaf, devils claw, chaste berry, yellow jasmine, black cohash, dong quai, piscidia erythrina (jamaican dogwood), viburnum and extracts and phytrochemicals derived therefrom, and combinations of these additives. While particular compounds or forms of a compound are listed above it should be recognized that it is contemplated that each of these materials can be provided as metabolites, derivatives, salts or other active forms of the listed material. In certain embodiments, the therapeutic compositions may specifically not be combined with folates.

In certain embodiments, regulated compounds or prescription drugs are preferably not used in combination with the therapeutic compositions of the present invention. Side effects of regulated compounds or prescriptions drugs may make them unappealing to combine with the therapeutic compositions of the present invention. As an example, topiramate, sold under the brand name TOPAMAX, is an anticonvulsant drug that may be used to treating migraines. The following side effects have been seen during clinical trials of topiramate: tiredness, pins and needles in the fingers and toes, dizziness, lowered sense of feeling in the skin, difficulty with language, nausea, diarrhea, indigestion, dry mouth, weight loss, decrease in appetite, drowsiness, forgetfulness, difficulty with concentration or attention, difficulty in sleeping (insomnia), anxiety, mood swings, depression, changes in taste and vision disorders. As such, in embodiments of the present invention, the therapeutic compositions are sold over-the-counter without regulation by avoiding combination with regulated compounds or prescription drugs.

The active ingredients of the present invention have been shown effective in published studies in placebo-controlled, peer-reviewed journals. In experimental formulations of this compound individuals have reported success with the compositions described herein. The combination of active ingredients may have unexpected synergistic effects as the ingredients work to prevent migraines through unique, hypothesized mechanisms of actions.

Use of the therapeutic composition may be combined with other non-pharmacologic prevention options to reduce the frequency and severity of migraine attacks. Other non-pharmacologic prevention options may include improving hydration, avoiding caffeine, preventing sleep pattern alterations, exercising regularly, relaxation training, thermal biofeedback combined with relaxation techniques, cognitive behavioral therapy, eating regularly, and others procedures.

Embodiments of the present invention may benefit headache suffers, offering a cost-effective approach to prevent migraines, thereby reducing the physical, psychological and social disability associated with migraines. Therapeutic compositions may be safe and may minimize drug interactions and/or adverse reactions.

Embodiments of the present invention may be formulated with one or both of regulated compounds/prescription drugs or supplements for various indications as indicated above. This may allow multiple variations based on secondary needs or specific requirements of physicians while maintaining a baseline effective combination supplement.

All the references cited herein are hereby incorporated by reference. It will be apparent to those skilled in the art that various modifications and variations can be made to the present invention without departing from the spirit or scope of the invention. Thus, it is intended that the present invention cover the modifications and variations of this invention provided they come within the scope of the appended claims and their equivalents. Accordingly, the invention is not limited by the embodiments described above which are presented as examples only, but can be modified in various ways within the scope of protection defined by the appended patent claims.

1. A therapeutic composition for preventing migraines, the therapeutic composition comprising:
riboflavin, magnesium, feverfew and coenzyme Q10.

2. The therapeutic composition of claim 1, further comprising, per day, approximately 100-400 mg riboflavin, approximately 150-600 mg magnesium, approximately 25-100 mg feverfew, and approximately 75-1200 mg coenzyme Q10, delivered as twice a day dosing.

3. The therapeutic composition of claim 2, further comprising, per dose, approximately 200 mg riboflavin, approximately 150 mg magnesium, approximately 25 mg feverfew, and approximately 75 mg coenzyme Q10.

4. The therapeutic composition of claim 1, wherein two doses are prescribed per day.

5. The therapeutic composition of claim 1, wherein a dosage form is one or more of a caplet, tablet, gel capsule, powder capsule, gel, liquid, or powder.

6. The therapeutic composition of claim 1, wherein the therapeutic composition does not comprise topiramate.

7. The therapeutic composition of claim 1, wherein the therapeutic composition does not comprise folates.

8. A therapeutic composition for preventing migraines, the therapeutic composition consisting essentially of: riboflavin, magnesium, feverfew and coenzyme Q10.

9. A method for preventing migraines, the method comprising:
administering a dose of a therapeutic composition; and
wherein the therapeutic composition comprises riboflavin, magnesium, feverfew and coenzyme Q10.

10. The method of claim 9, comprising, per day, approximately 100-400 mg riboflavin, approximately 150-600 mg magnesium, approximately 25-100 mg feverfew, and approximately 75-1200 mg coenzyme Q10, delivered as twice a day dosing.

11. The method of claim 10, further comprising, per dose, approximately 200 mg riboflavin, approximately 150 mg magnesium, approximately 25 mg feverfew, and approximately 75 mg coenzyme Q10.

12. The method of claim 9, further comprising administering two doses per day.

13. The method of claim 9, wherein a dosage form is one or more of a caplet, tablet, gel capsule, powder capsule, gel, liquid, or powder.

14. The method of claim 9, wherein the therapeutic composition does not comprise topiramate.

15. The method of claim 9, wherein the therapeutic composition does not comprise folates.
